(19) United States
(12) Patent Application Publication
(10) Pub. No.: US 2011/0003760 A1
(43) Pub. Date: Jan. 6, 2011

(54) ABNORMAL PROTEIN REMOVING METHOD

(75) Inventors: Satoshi MIYATA, Yokohama (JP); Yukari UMINO, Yokohama (JP)

Correspondence Address: KNOBBE MARTENS OLSON & BEAR LLP, 2040 MAIN STREET, FOURTEENTH FLOOR, IRVINE, CA, 92614, US

(73) Assignee: FANCL CORPORATION, Yokohama-shi (JP)

(21) Appl. No.: 12/883,408

(22) Filed: Sep. 16, 2010

Related U.S. Application Data
(62) Division of application No. 12/088,919, filed on Apr. 1, 2008.

(30) Foreign Application Priority Data
Oct. 3, 2005 (JP) 2005-289491

(51) Int. Cl. A61K 8/60; A61K 31/704; A61P 17/00; A61Q 19/00; A61Q 19/08

(52) U.S. Cl. 514/26

(57) ABSTRACT

A method for treating symptoms or diseases associated with accumulation of abnormal protein in the body includes administrating to a subject desiring such treatment a composition containing silybin and soybean saponin in an amount effective to reduce or remove abnormal protein produced in the body.

### CROSS REFERENCE TO RELATED APPLICATIONS

This is a divisional application of U.S. patent application Ser. No. 12/088,919, field Apr. 1, 2008, which is the U.S. National Phase under 35 U.S.C. §371 of International Application PCT/JP2006/314403, filed Jul. 20, 2006, which claims priority to Japanese Patent Application No. 2005-289491, filed Oct. 3, 2005. The International Application was published under PCT Article 21(2) in a language other than English. The disclosure of the U.S. patent application is herein incorporated by reference in its entirety.

### BACKGROUND OF THE INVENTION

1. Technical Field

The present invention relates to a composition for removing abnormal protein as well as a purpose of use of the same.

2. Prior Art

Various cases of oxidation damage caused by active oxygen to cells and tissues in the body have been presenting problems of late. Active oxygen is highly reactive and destroys various components that make up the body, and an involvement of active oxygen in a wide range of diseases, such as Alzheimer's disease, Parkinson's disease, dementia with Lewy bodies, triplet repeat disease, amyotrophic lateral sclerosis, cataract, arteriosclerosis, diabetic nephropathy, cerebral apoplexy, myocardial infarction, rheumatism, cancer, gastric ulcer, and skin conditions including lines, sagging, dull complexion and pigmentation, has been shown (Non-patent Literature 1). Factors that are known to increase active oxygen include aging, excessive exercise, exposure to ultraviolet (UV) light, and mental stress. When active oxygen increases, oxidized protein, or so-called abnormal protein, accumulates in the body and this accumulation of abnormal protein triggers various diseases such as those mentioned above (Non-patent Literature 2). The skin is particularly vulnerable to oxidation damage caused by exposure to UV light, and it is known that exposure to UV light causes DNA damage in epidermal keratinocytes and skin fibroblasts, as well as breakdown of elastin and collagen that support the skin's elasticity, thereby promoting formation of lines and pigmentation (Non-patent Literature 3).

To prevent oxidation damage caused by active oxygen, attempts have been made to eliminate active oxygen in the body, and thereby suppress protein oxidation, by means of ingestion and application of anti-oxidants. Representative anti-oxidants include tocopherols, carotinoids, and flavonoids, and some of these substances are currently in use in foods and cosmetics.

However, while ingesting and applying anti-oxidants has some effect on the elimination of active oxygen produced in the body, it has no effect at all on the elimination of abnormal protein already accumulated in the body. Accordingly, removal of accumulated abnormal protein is essential in the improvement of various diseases in which abnormal protein accumulated in the body has a hand.

Proteasome is known as an enzyme that removes abnormal protein in the body. Proteasome is a giant multi-component complex having an intricate molecular structure, and its physiological function in the body has been a target of active research in recent years. Proteasome not only removes abnormal protein resulting from abnormal folding or molecular association in the process of formation of the three-dimensional protein structure, to implement quality control of protein, but it also removes protein that has been denatured or damaged due to exposure to UV light, oxidation stress, etc., and thus it is closely involved in the body's mechanism to cope with stress (Non-patent Literature 4). In the skin, it is known that proteasome activity drops and oxidized collagen increases with age (Non-patent Literature 5). As explained above, proteasome is a substance that plays a central role in the maintenance and monitoring of homeostasis of cells through removal of abnormal protein.

In view of the above, compositions that promote proteasome activity in the body and thereby prevent and improve various diseases have been developed. Example of such substances that have been developed to date include a proteasome activity promoter containing Ganoderma lucidum extract (Patent Literature 1), proteasome action enhancer containing a specific peptide compound (Patent Literature 2), composition for removing abnormal protein that contains soybean-derived saponin having an action to promote proteasome activity (Patent Literature 3), and composition for promoting proteasome activity that contains kale and/or extract therefrom (Patent Literature 4).
Patent Literature 1: Japanese Patent Laid-open No. 2002-29996
Patent Literature 2: International Patent Publication No. WO00/04042
Patent Literature 3: Japanese Patent Laid-open No. 2002-179592
Patent Literature 4: Japanese Patent Laid-open No. 2004-91398
Patent Literature 5: Japanese Patent Laid-open No. Hei 5-286864
Patent Literature 6: Japanese Patent No. 2948818
Patent Literature 7: Japanese Patent Laid-open No. 2000-169328
Patent Literature 8: Japanese Patent Laid-open No. 2000-169332
Patent Literature 9: Japanese Patent Application No. 2002-255448
Patent Literature 10: Examined Japanese Patent Laid-open No. Hei 5-9406
Patent Literature 11: International Patent Publication No. WO2004/085429
Patent Literature 12: Examined Japanese Patent Laid-open No. Sho 63-41396
Patent Literature 13: Japanese Patent Laid-open No. 2004-115438
Patent Literature 14: Japanese Patent Laid-open No. 2004-131431
Non-patent Literature 1: Roka no Mekanizumu to Seigyo (Mechanism and Control of Aging), Daisaburo Fujimoto (ed.), IPC Ltd., Jun. 30, 1993
Non-patent Literature 2: BIO Clinica, Vol. 11, No. 5, 1996
Non-patent Literature 3: Keshohin no Yuyosei: Hyoka Gijutsu no Shimpo to Shorai Tembo (Utility of Cosmetics: Progress and Future Perspective of Evaluation Technologies), Society of Cosmetic Chemists of Japan (ed.), Yakuji Nippo, 2001, Kyoritsu Press
Non-patent Literature 4: Tampakushitsu Kakusan Koso (Protein, Nucleic Acid and Enzyme), Vol. 44, No. 6, pp. 766-775, 1999, Kyoritsu Press
Non-patent Literature 5: Journal of Gerontology 2000, Vol. 55 (5), pp. 220-227
Non-patent Literature 6: Tennen Yakubutsu Jiten (Encyclopedia of Natural Medicine), Takuo Okuda (ed.), Hirokawa Shoten, Mar. 3, 1986
Non-patent Literature 7: Wagner, H., et al., Arznein. Forsch, 18, 696, 1968
Non-patent Literature 8: Wagner, H., et al., Arznein. Forsch, 24, 466, 1974
Non-patent Literature 9: Tittel, G., et al., J. Chromatogr., 135, 499, 1977
Non-patent Literature 10: Tittel, G., et al., J. Chromatogr., 153, 227, 1978
Non-patent Literature 11: Quercia, V., et al., Chromatography in Biochemistry, Medicine and Environmental Research, Frigerio A. (ed.), Elsevier Scientific Publishing Company, Amsterdam, 1983, p. 1
Non-patent Literature 12: Nam-Cheol, Kim, et al., Complete isolation and characterization of silybins and isosilybins from milk thistle (Silybum marianum), Org. Biomol. Chem., 2003, 1, 1684-1689
Non-patent Literature 13: Agric Biol Chem, 55, 315-322, 1991
Non-patent Literature 14: Agric Biol Chem, 57, 546-550, 1993
Non-patent Literature 15: Kiso to Rinsho (Clinical Report), Vol. 15, No. 5, 1981

### SUMMARY OF THE INVENTION

### Problems to Be Solved by the Invention

It is an object of the present invention to provide a composition capable of efficiently removing abnormal protein produced in living tissues, especially skin, due to exposure to UV light, etc.

### Means for Solving the Problems

To achieve the aforementioned object, the inventors turned to various plant-derived compounds and plant extracts in search of components that promote proteasome activity and thereby suppress accumulation of oxidized protein in the body, especially oxidized protein whose production increased due to exposure to UV light. As a result, silybin, a component derived from Silybum marianum, was identified as a plant-derived compound, while Bletilla striata extract and Iris sanguinea extract were identified as plant extracts, having desired effects. In addition, it was found that using silybin together with Bletilla striata extract or soybean saponin would promote proteasome activity and remove oxidized protein in a synergistic manner, the discovery of which led to the present invention. Abnormal collagen was confirmed as one form of abnormal protein targeted by the present invention.

The key points of the present invention are as follows:
(1) A composition for removing abnormal protein containing one or two or more substances selected from silybin, Bletilla striata extract and Iris sanguinea extract.
(2) A composition for removing abnormal protein according to (1), characterized in that the abnormal protein is abnormal collagen.
(3) A composition for promoting proteasome activity containing one or two or more substances selected from silybin, Bletilla striata extract and Iris sanguinea extract.
(4) A composition for removing abnormal protein according to (1) or (2) or a composition for promoting proteasome activity according to (3), characterized by containing silybin and Bletilla striata extract.
(5) A composition for removing abnormal protein according to (1) or (2) or a composition for promoting proteasome activity according to (3), characterized by containing silybin and soybean saponin.
(6) A composition for preventing and/or improving lines, sagging, dull complexion and pigmentation that contains any composition according to any one of (1) to (5).
(7) A composition according to any one of (1) to (6) provided as a preparation for external use.
(8) A cosmetic characterized by containing silybin and Bletilla striata extract and/or soybean saponin.
(9) A food characterized by containing silybin and Bletilla striata extract and/or soybean saponin.
(10) A composition according to (7) or (9) for animals.

### EFFECTS OF THE INVENTION

By using a composition conforming to the present invention, abnormal protein can be removed or suppressed. As a form of abnormal protein, abnormal collagen that increases with age (such as oxidized collagen) can be efficiently removed and therefore the present invention is effective in the improvement of signs of aging skin such as lines, sagging and dull complexion. Also, proteasome activity can be promoted by using a composition conforming to the present invention.

In other words, a composition conforming to the present invention is effective in the prevention or treatment of diseases or disorders caused by protein breakdown abnormality (Alzheimer's disease, Parkinson's disease, dementia with Lewy bodies, triplet repeat disease, amyotrophic lateral sclerosis, cataract, arteriosclerosis, diabetic nephropathy, aging of skin due to exposure to light, and skin conditions such as lines, sagging, dull complexion and pigmentation). Furthermore, it is also useful as an anti-aging cosmetic or food.

To be specific, the present invention is expected to present an anti-aging effect as well as effects to suppress lines, sagging, dull complexion, pigmentation and skin damage caused by UV light.

Specific applications include cosmetics and foods, among others. The present invention can also be used for animals such as pets.

### BRIEF DESCRIPTION OF THE DRAWINGS

FIG. 1 A graph showing the actions, exhibited by various plant extracts, of suppressing the accumulation of carbonylated protein in skin fibroblasts under conditions with and without UV irradiation, presented as detection results by Western blotting in a reducing condition

FIG. 2 A graph showing the actions, exhibited by various plant extracts, of suppressing the accumulation of carbonylated protein in skin fibroblasts under conditions with and without UV irradiation, presented as detection results by Western blotting in a non-reducing condition

FIG. 3 A graph showing the actions, exhibited by Bletilla striata extract, of suppressing the accumulation of carbonylated protein in a three-dimensional human skin model under conditions with and without UV irradiation, presented as detection results by Western blotting in a reducing condition

FIG. 4 A graph showing the actions, exhibited by Bletilla striata extract, of suppressing the accumulation of carbonylated protein in a three-dimensional human skin model under conditions with and without UV irradiation, presented as detection results by Western blotting in a non-reducing condition

FIG. 5 A graph showing the actions, exhibited by silybin, retinoic acid and retinol, of suppressing the accumulation of carbonylated protein in a three-dimensional human skin model under conditions with and without UV irradiation, presented as detection results by Western blotting in a reducing condition

FIG. 6 A graph showing the actions, exhibited by silybin, retinoic acid and retinol, of suppressing the accumulation of carbonylated protein in a three-dimensional human skin model under conditions with and without UV irradiation, presented as detection results by Western blotting in a non-reducing condition

FIG. 7 A graph showing the actions, exhibited by silybin, Bletilla striata extract and soybean saponin, of suppressing the accumulation of carbonylated protein in a three-dimensional human skin model under conditions with and without UV irradiation, presented as detection results by Western blotting in a non-reducing condition

FIG. 8 A graph showing the actions, exhibited by silybin, Bletilla striata extract and soybean saponin, of suppressing the accumulation of carbonylated collagen in a three-dimensional human skin model under conditions with and without UV irradiation, presented as detection results by Western blotting in a non-reducing condition

FIG. 9 A graph showing the actions, exhibited by various plant extracts, of promoting proteasome activity in skin fibroblasts under conditions with and without UV irradiation

FIG. 10 A graph showing the actions, exhibited by Bletilla striata extract, of promoting proteasome activity in a three-dimensional human skin model under conditions with and without UV irradiation

FIG. 11 A graph showing the actions, exhibited by silybin, retinoic acid and retinol, of promoting proteasome activity in a three-dimensional human skin model under conditions with and without UV irradiation

FIG. 12 A graph showing the actions, exhibited by silybin, Bletilla striata extract and soybean saponin, of promoting proteasome activity in a three-dimensional human skin model under conditions with and without UV irradiation

### BEST MODE FOR CARRYING OUT THE INVENTION

The present invention is explained below in details.

In the context of the present invention, abnormal protein generally refers to protein that has been oxidized or saccharified or aldehyde-modified, or misfolded protein, with age.

Silymarin (CAS No. 65666-07-1) is a general term for flavonolignans extracted from milk thistle of the Compositae family (scientific name: Silybum marianum Gaertn; other names: Mary thistle, cotton thistle; CAS No. 84604-20-6), and expressed by the molecular formula C₂₅H₂₂O₁₀. It is a composition that contains silybin (CAS No. 22888-70-6), silydianin (CAS No. 29782-68-1), silychristin (CAS No. 33889-69-9), isosilybin (CAS No. 72581-71-6), etc. (Non-patent Literature 6). Under the present invention, compositions that contain these flavonolignans contained in milk thistle extract are collectively referred to as silymarin, as is done in the prior art. Also, silymarin is a mixture of flavonolignans, as mentioned above, and the content of silymarin in a plant extract or plant can be measured using any method based on spectrophotometry (Non-patent Literature 7), method using thin-layer chromatography (Non-patent Literature 8), or method using high-speed liquid chromatography (Non-patent Literatures 9 to 11). Among these measurement methods, 2,4-dinitrohydrazine analysis, which is a method based on spectrophotometry, is reported in the German Pharmacopeia (monograph relating to Silybum marianum fruit) and widely used. Accordingly, the 2,4-dinitrohydrazine analysis method is used to quantify compositions constituted by a mixture of the aforementioned ingredients where, specifically, quantities are indicated by equivalent silymarin percentages by mass.

In Europe, silymarin has been used since ancient times for the purpose of preventing and treating liver diseases. Silymarin is also widely known as an anti-oxidant. Silymarin is known as a composition having beneficial effects on skin and the known beneficial effects of silymarin include a utility as a treatment drug for psoriasis and atopic dermatitis (Patent Literature 5), utility as a composition containing a flavonolignan and phospholipid complex as its active ingredient and therefore useful in the treatment of erythema, burns, dystrophy of skin or viscous membrane and dermatitis, prevention of skin aging, and protection of skin against external irritations such as radioactive ray, wind and sunlight (Patent Literature 6), utility as an agent to enhance the skin's permeation-blocking barrier property (Patent Literature 7), utility as an agent to suppress sebum secretion (Patent Literature 8), utility as a composition to prevent aging of skin by preventing and improving flattening of epidermis (Patent Literature 9), utility as a cosmetic to prevent aging of skin by exhibiting an anti-oxidative property (Patent Literature 10), and action to promote type I collagen production and elastin production (Patent Literature 11), among others.

However, the action of silymarin to promote proteasome activity and thereby suppress accumulation of abnormal protein has not been known. The present invention shows that use of silymarin leads to suppression of accumulation of abnormal protein as well as removal of accumulated abnormal protein and that, by using silymarin, especially silybin, and Bletilla striata extract or soybean saponin in combination, the aforementioned effect increases synergistically compared to when they are used alone.

As ways to isolate silymarin from milk thistle fruit at high purity, a method to isolate silymarin with a purity of 70 to 80% and a method to isolate silymarin with a purity of 90 to 96% (Patent Literature 12) have been reported. Silymarin is normally extracted from milk thistle seed using ethanol, ethyl acetate, acetone, etc., and is sold commercially as an extract material in the form of dry powder produced by spray-drying an extract. As for silybin that can be used under the present invention, any such silybin prepared and sold commercially in this manner can be used directly. It is also possible to use a concentrated extract of silybin from milk thistle using a traditional method, or any compound produced by isolating and refining such extract can be used, as well (Non-patent Literature 12). Furthermore, milk thistle extract and other plant extracts containing silybin can be used, or silymarin can be used as silybin.

Soybean-derived saponin is widely found in seed coat, seed leaf and hypocotyl of soybean seed or leaf, stem, root and other parts of soybean plant. Structurally soybean-derived saponin is similar to glycyrrhizin, but it has a sugar chain comprising two to five sugars in the triterpenoid skeleton. Soybean saponin is classified into four groups (groups A, B, E and DDMP) based on the structure of aglycon (non-sugar part), and saponin varieties in all these groups have various sugar chain structures. To date, eight types of group A saponin, two types of group E saponin, five types of group B saponin and six types of group DDMP saponin have been identified, characterized by having agricon structures of Soyasapogenol A, B, E and DDMP, respectively (Non-patent Literatures 13 and 14).

Prior to the present invention, the inventors had already focused on the pharmacological actions of soybean saponin and studied them continuously, ultimately revealing such functions of soybean saponin as removal of abnormal protein (Patent Literatures 3 and 13) and prevention or improvement of damage caused by UV light (Patent Literature 14), among others. In developing the present invention, the inventors build upon these previous studies and found that by using silymarin, especially silybin, and soybean saponin in combination, the effect of suppressing accumulation of abnormal protein increases synergistically compared to when they are used alone.

Soybean-derived saponin used in the present invention includes all types of soybean-derived saponin as mentioned above, and soybean-derived saponin used in the present invention may also be a substance that contains a large amount of a specific type of soybean-derived saponin. Soybean-derived saponin used in the present invention may be in a dry powder form, or in a form dissolved in an organic solvent such as ethanol or dimethyl sulfoxide.

In general, many forms of saponin exhibit hemolytic property. However, soybean saponin has been reported to present little hemolytic property (Non-patent Literature 15). Also, the inventors measured the hemolytic index of soybean saponin relative to a 2% rabbit blood suspension and found that the measured hemolytic index was 100 or less, equivalent to the hemolytic index of carrot saponin, and thus hemolytic property was absent in soybean saponin in agreement with other reports. Also, the inventors conducted a separate test on mutagenicity and acute toxicity of group B soybean saponin to examine its safety, and found that group B had no problem in either property and was very safe.

Bletilla striata is a perennial belonging to the Orchidaceae family that grows naturally in swamps and on cliffs, etc. Compared to other members of the Orchidaceae family, Bletilla striata is exceptionally easy to grow and very fertile. Rhizoma Bletillae, which is a type of Chinese medicine, is made by steaming or boiling and then drying Bletilla striata root and stem, and is used as a hemostatic drug, pus discharge drug, demulcent drug, and emollient, to be administered internally or externally to treat expectoration of blood, vomiting of blood, nose bleed, cuts, burns, and swellings (Non-patent Literature; Tennen Yakubutsu Jiten (Encyclopedia of Natural Medicine), Takuo Okuda (ed.), Hirokawa Shoten, p. 355). Bletilla striata is known to have an anti-oxidative action (Japanese Patent Laid-open No. 2002-205933), utility as a Maillard reaction inhibitor (Japanese Patent Laid-open No. Hei 11-106336), and whitening action (Japanese Patent No. 3233776). However, its action to promote proteasome activity and thereby suppress accumulation of abnormal protein has not been known. The present invention shows that, by using silybin and Bletilla striata extract in combination, the effect of suppressing accumulation of abnormal protein increases synergistically compared to when they are used alone.

Iris sanguinea is a perennial that grows widely in East Asia including Japan (from Hokkaido to Kyushu), and in many cases the natural habitat of Iris sanguinea is dry field in the sun in mountains. Since its root and stem contain flavoayamenin, Iris sanguinea is used to treat dermatomycosis as well as inflammation, abdominal pain and stomach ache. Iris sanguinea is known to eliminate hydrogen peroxide (Japanese Patent Laid-open No. 2001-131046). However, its action to promote proteasome activity and thereby suppress accumulation of abnormal protein has not been known. The present invention shows that Iris sanguinea extract has an action to promote proteasome activity and thereby suppress accumulation of abnormal protein.

Under the present invention, all parts of plants containing silybin, Bletilla striata and Iris sanguinea can be used, including leaf, stem, sprout, flower, wooden part, bark and other parts that grow on the ground; root, tube and other parts that grow underground; as well as seed and resin.

Under the present invention, silybin and any plant containing silybin, Bletilla striata, Iris sanguinea and soybean saponin can be used in a dry form produced by drying each substance directly, or in a dissolved form produced by dissolving each substance in various solvents. For example, the above substances can be dissolved in water; ethanol, methanol and other alcohols; propylene glycol, 1,3-butylene glycol and other polyhydric alcohols; as well as ether, acetone, ethyl acetate and other organic solvents.

Under the present invention, silybin and any plant containing silybin, Bletilla striata and Iris sanguinea can be naturally dried, dried by hot air, freeze-dried, or fermented and the obtained dried or fermented substance can be used directly. If a plant extract is to be prepared, the result obtained through extraction, concentration, pulverization or other process according to normal methods can be used.

Today, accumulated abnormal protein is known to have a hand in a wide range of diseases, such as Alzheimer's disease, Parkinson's disease, dementia with Lewy bodies, triplet repeat disease, amyotrophic lateral sclerosis, cataract, arteriosclerosis, diabetic nephropathy, cerebral apoplexy, myocardial infarction, rheumatism, cancer, gastric ulcer, and signs of aging skin including lines, sagging, dull complexion and pigmentation. Accordingly, it is suggested that ingesting a composition for removing abnormal protein as proposed by the present invention would make it possible to prevent or treat the aforementioned diseases. In particular, the present invention is expected to prevent or improve signs of aging skin such as lines, sagging, dull complexion and pigmentation, which should contribute to maintenance of youthful skin.

A composition conforming to the present invention is useful as a cosmetic or health food that prevents aging, as an anti-aging cosmetic or beauty food, or as a cosmetic or health food that prevents rust. The composition for removing abnormal protein as proposed by the present invention has excellent effects in mammals and is also very safe.

A composition conforming to the present invention effectively breaks down denatured protein (abnormal protein) that has been produced in cells due to active oxygen generated by exposure to UV light. Accordingly, it has the effect of suppressing cell damage caused by exposure to UV light. In other words, the present invention is useful as a composition for preventing or improving UV damage, capable of preventing or improving UV damage suffered by living tissues, especially skin tissues, which are being exposed to or have been exposed to UV light.

A composition containing silybin or silymarin, Bletilla striata extract, Iris sanguinea extract and/or soybean saponin as its main ingredient, as proposed by the present invention, can be manufactured as a cosmetic or external drug for skin, or food.

When the present invention is used as a cosmetic, silybin, silymarin, or plant or plant extract containing silybin can be used directly as an ingredient, or it can be added to a wheat germ oil or olive oil and the resulting oil mixture can be used as an ingredient, in the manufacture of a cosmetic.

When the present invention is used as a food, silybin, silymarin, or plant or plant extract containing silybin can be used directly as a food, or it can be mixed with various nutritional components to produce a food, or added to a desired food. For example, it is possible to add an appropriate auxiliary such as starch, milk sugar, maltose, vegetable oil powder, cacao powder or stearic acid, and then shape the mixture into an edible form, such as granule, pellet, tablet, capsule or paste, using a commonly used method to produce a health supplement, functional health food or the like. In addition, any of the aforementioned materials can also be added to various food products, such as ham, sausage and other processed meat products, fish cake and other processed seafood products, bread, confectionary, butter, powder milk and fermented milk products, or it can be added to water, fruit juice, milk, soda and other beverages.

The effective content of silybin or silymarin, Bletilla striata extract, Iris sanguinea extract and/or soybean saponin in the composition is not at all limited, but it is instead selected and determined as deemed appropriate according to the preparation method and dosage form of each component, among other conditions. When using silybin and/or soybean saponin in an external drug for skin, however, desirably the silybin and/or soybean saponin content should be 0.01 to 2 percent by weight. When using Bletilla striata extract and/or Iris sanguinea extract in an external drug for skin, on the other hand, desirably the Bletilla striata extract and/or Iris sanguinea extract content should be 0.1 to 5 percent by weight.

When using silybin and/or soybean saponin in a tablet, drink or other forms of food, desirably the silybin and/or soybean saponin content should be 0.1 to 10 percent by weight. When using Bletilla striata extract and/or Iris sanguinea extract in a tablet, drink or other forms of food, on the other hand, desirably the Bletilla striata extract and/or Iris sanguinea extract content should be 1 to 20 percent by weight.

Under the present invention, the effective application amount of the component that contains silybin or silymarin, Bletilla striata extract, Iris sanguinea extract and/or soybean saponin as its main ingredient can be determined as deemed appropriate based on the application pathway, application schedule and dosage form, among other conditions. For example, a composition that contains silybin, Bletilla striata extract, Iris sanguinea extract and/or soybean saponin as its main ingredient can be taken by an appropriate amount adjustable within a range of 0.01 to 10 g per day, with the amount taken all at once or in portions over several times.

In food applications, the composition can be used directly or by adding various nutritional components. For example, it is possible to add an appropriate auxiliary such as starch, milk sugar, maltose, vegetable oil powder, cacao powder or stearic acid, and then shape the mixture into an edible form, such as granule, pellet, tablet, capsule or paste, using a commonly used method to produce a health supplement, functional health food or other edible form, or the composition can also be added to various food products, such as ham, sausage and other processed meat products, fish cake and other processed seafood products, bread, confectionary, butter, powder milk and fermented milk products, or it can be added to water, fruit juice, milk, soda and other beverages. Such agents and foods may be manufactured using preparation technologies that are normally employed.

In cosmetic applications, the composition can be used directly as an ingredient, or it can be added to a wheat germ oil or olive oil and the oil mixture can be used as an ingredient, in the manufacture of a cosmetic.

A parenteral composition can be applied in the form of liquid such as aqueous solution, oil solution, emulsion or suspension; in the form of semi-solid such as gel or cream; or in the form of solid such as powder, granule, capsule, microcapsule or solid. Any of these forms can be prepared using a known traditional method for use as a lotion, emulsion, gel, cream, ointment, plaster, poultice, aerosol, suppository, injection, powder or various other dosage forms. These can be spread, attached, sprayed or otherwise applied to the body. Among these dosage forms, lotion, emulsion, cream, ointment, plaster, poultice and aerosol are especially suitable as a form of external drug for skin. As for cosmetics, the present invention can be made into skin care products such as lotion, milky lotion, cream and mask; makeup products such as makeup base lotion, makeup cream, milky or cream-type or paste-type foundation, lipstick, eye color and cheek color; and body care products such as hand cream, leg cream and body lotion, among others.

For greater convenience, the present invention can be produced in the form of a composition mixed with an extender. Examples of substances that can be used as an extender include glucose, lactose, maltose, sucrose and other sugars; sorbitol and other sugar alcohols; dextrin, cyclodextrin and other processed starches; wheat starch, cornstarch and other starches; casein, soybean protein and other proteins; Arabian gum, sodium alginate, sodium caseinate, gelatin, pectin, powder cellulose, carboxymethyl cellulose and other polymer stabilizers; lecithin, sucrose fatty acid ester, propylene glycol fatty acid ester, glycerin fatty acid ester and other emulsifiers; and calcium powder, among others.

The composition for removing abnormal protein as proposed by the present invention can contain a compound having anti-oxidative property, in addition to the aforementioned silybin, Bletilla striata extract, Iris sanguinea extract and/or soybean saponin. This compound having anti-oxidative property is not limited in any way, but examples include, among others, various vitamins, various polyphenols such as silymarin, tocotrienol, coenzyme Q10 and natural components containing any of the foregoing.

The composition for removing abnormal protein as proposed by the present invention can contain a compound having the effect of promoting biocollagen synthesis, in addition to the aforementioned silybin, Bletilla striata extract and soybean saponin. This compound having the effect of promoting biocollagen synthesis is not limited in any way, but examples include, among others, collagen and gelatin decomposition products as well as peptide mixtures having a tripeptide containing glycine at the N end.

For collagen, collagen extract from skin, bone, tendon and other connective tissues of cow, pig, fish, etc., or gelatin made by denaturing collagen under heat, or any other form of collagen can be used. Among collagen and/or gelatin decomposition products, desirably a polypeptide containing those with a molecular weight of 400 or less should be used. A polypeptide containing those with an average molecular weight of around 200 to 300 by a large quantity is more preferable. A polypeptide containing those with a molecular weight of 400 or less, or containing those with an average molecular weight of around 200 to 300 by a large quantity, has an amino acid molecular weight of around 100 and therefore such polypeptide is considered to contain a tripeptide by a large quantity. A collagen and/or gelatin decomposition product with a molecular weight of 400 or less can be refined, but not refining it will present no problem at all. For example, it can be mixed with other collagen and/or gelatin decomposition product, etc.

On the other hand, a collagen and/or gelatin decomposition product, when it contains a peptide with a molecular weight of approx. 400 or less as a specific effective ingredient, can improve the collagen synthesis promotion activity in the body as a result of hydrolysis of such peptide.

The composition for removing abnormal protein as proposed by the present invention can be used for the purpose of preventing aging or UV damage. Furthermore, such composition, when it contains a compound exhibiting an action to remove abnormal protein, along with a compound exhibiting an anti-oxidative action or compound exhibiting an action to promote biocollagen synthesis, provides an anti-aging composition capable of preventing accumulation of abnormal protein and removing accumulated abnormal protein. It has also been confirmed that the present invention can provide an anti-aging composition having the function to prevent accumulation of abnormal collagen as one type of abnormal protein, and also remove accumulated abnormal collagen.

The compound having an action to remove abnormal protein can be used as a cosmetic. This cosmetic can be used to protect the skin against lines, sagging, dull complexion or pigmentation or to moisturize the skin. If designed not as an oral or hemolytic composition, a composition conforming to the present invention can be administered as an injection or in other parenteral form. If administered orally, such composition may be administered in the form of food, such as health food or beauty food.

A composition conforming to the present invention can be applied in the form of liquid such as aqueous solution, oil solution, emulsion or suspension; in the form of semi-solid such as gel or cream; or in the form of solid such as powder, granule, tablet or capsule. A known traditional method can be used to prepare the composition into any of these forms for use in various dosage forms. Among these dosage forms, lotion, emulsion, cream, ointment, plaster, poultice and aerosol are suitable as a form of external drug for skin.

A cosmetic conforming to the present invention can contain vegetable oil and other oils, higher fatty acids, higher alcohols, silicones, anionic surface active agents, cationic surface active agents, ampholytic surface active agents, nonionic surface active agents, preservatives, sugars, sequestering agents, water-soluble polymers and other polymers, thickeners, powder components, UV absorbents, UV blockers, hyaluronic acids and other moisture-keeping agents, aromatic substances, pH regulators, and others. It can also contain vitamins, skin activators, blood-circulation promoters, agents for controlling normal bacteria flora, active oxygen removers, anti-inflammatory agents, anti-cancer agents, whitening agents, anti-bacterial agents and other medicinal and bioactive components.

As for cosmetics, the present invention can be made into skin care products such as lotion, milky lotion, cream and mask; makeup products such as makeup base lotion, makeup cream and milky or cream-type or paste-type foundation; body care products such as hand cream, leg cream and body lotion; bath agents; and hair care products, among others. These dosage forms can be manufactured according to any preparation method normally used in the manufacture of cosmetics. The present invention can also be made into such makeup products as lipstick, eye color and cheek color.

In addition to the above, other substances such as those listed below may be added according to the purpose and dosage form.

Examples of oils that can be used include, among others, camellia oil, evening primrose oil, Macadamia nut oil, olive oil, rape seed oil, corn oil, sesame oil, jojoba oil, germ oil, wheat germ oil, glycerin trioctate and other liquid oils; cacao oil, coconut oil, hardened coconut oil, palm oil, palm kernel oil, tallow oil, tallow kernel oil, hardened oil, hardened castor oil and other solid oils; and bees wax, candelilla wax, cotton wax, rice bran wax, lanolin, lanolin acetate, liquid lanolin, sugarcane wax and other waxes.

Examples of carbohydrates that can be used include, among others, liquid paraffin, squalene, squalane and micro crystalline wax.

Examples of higher fatty acids that can be used include, among others, lauric acid, myristic acid, palmitic acid, stearic acid, oleic acid, linoleic acid, linolenic acid, docosa-hexaenoic acid (DHA) and eicosa-pentaenoic acid (EPA).

Examples of higher alcohols that can be used include, among others, lauryl alcohol, stearyl alcohol, cetyl alcohol, cetostearyl alcohol and other straight-chain alcohols; and monostearyl glycerin ether, lanolin alcohol, cholesterol, phytosterol, octyl dodecanol and other branched-chain alcohols.

Examples of silicones that can be used include, among others, dimethyl polysiloxane, methyl phenyl polysiloxane and other chain polysiloxanes; and decamethyl cyclopentane siloxane and other cyclic polysiloxanes.

Examples of anionic surface active agents that can be used include, among others, sodium laurate and other fatty acid salts; sodium lauryl sulfate and other higher alkyl sulfuric ester salts; POE triethanol amine lauryl sulfate and other alkyl ether sulfuric ester salts; N-acyl sarcosinic acid; sulfosuccinic acid salt; and N-acyl amino acid salt.

Examples of cationic surface active agents that can be used include, among others, stearyl trimethyl ammonium chloride and other alkyl trimethyl ammonium salts; and benzalkonium chloride; and benzethonium chloride.

Examples of ampholytic surface active agents that can be used include, among others, alkyl betaine, amide betaine and other betaine surface active agents.

Examples of nonionic surface active agents that can be used include, among others, sorbitan monooleate and other sorbitan fatty acid esters; and hardened castor oil derivatives.

Examples of preservatives that can be used include, among others, methyl paraben and ethyl paraben.

Examples of sequestering agents that can be used include, among others, ethylene diamine disodium tetraacetate, edetic acid, sodium edetate salt and other edetic acid salts.

Examples of polymers that can be used include, among others, Arabian gum, tragacanth gum, galactan, guar gum, carrageenan, pectin, agar, quince seed, dextran, pullulan, carboxy methyl starch, collagen, casein, gelatin, methyl cellulose, methyl hydroxy propyl cellulose, hydroxy ethyl cellulose, carboxy methyl cellulose sodium (CMC), sodium alginate, carboxy vinyl polymers (CARBOPOL, etc.) and other vinyl polymers.

Examples of thickeners that can be used include, among others, carrageenan, tragacanth gum, quince seed, casein, dextrin, gelatin, CMC, hydroxy ethyl cellulose, hydroxy propyl cellulose, carboxy vinyl polymer, guar gum, xanthan gum and bentonite.

Examples of powder components that can be used include, among others, talc, kaolin, mica, silica, zeolite, polyethylene powder, polystyrene powder, cellulose powder, inorganic white pigments, inorganic red pigments, titanium oxide coated mica, titanium oxide coated talc, colored titanium oxide coated mica and other pearl pigments; and red 201, red 202 and other organic pigments.

Examples of UV absorbents that can be used include, among others, para-aminobenzoic acid, phenyl salicylate, isopropyl para-methoxy cinnamate, octyl para-methoxy cinnamate and 2,4-dihydroxy benzophenone.

Examples of UV blockers that can be used include, among others, titanium oxide, talc, carmine, bentonite, kaolin and zinc oxide.

Examples of moisture-keeping agents that can be used include, among others, polyethylene glycol, propylene glycol, dipropylene glycol, 1,3-butylene glycol, 1,2-pentane diol, glycerin, diglycerin, polyglycerin, xylitol, maltitol, maltose, sorbitol, glucose, fructose, sodium chondroitin sulfate, sodium hyaluronate, sodium lactate, pyrrolidone carboxylic acid and cyclodextrin.

Examples of medicinal components that can be used include, among others, various forms of vitamins including vitamin A oil, retinol and other forms of vitamin A; riboflavin and other forms of vitamin B₂; pyridoxine hydrochloride and other forms of vitamin B₆; L-ascorbic acid, L-ascorbic acid ester phosphate, L-ascorbic acid ester monopalmitate, L-ascorbic acid ester dipalmitate, L-ascorbic acid-2-glucoside and other forms of vitamin C; calcium pantothenate and other pantothenates; vitamin D₂, cholecalciferol and other forms of vitamin D; α-tocopherol, tocopherol acetate, nicotinic acid DL-α-tocopherol and other forms of vitamin E.

Other examples of substances that can be added include, among others, placenta extract, glutathione, Saxifraga stolonifera extract and other whitening agents; royal jelly, beech extract and other skin activators; capsaicin, zingherone, cantharides tincture, ichthammol, caffeine, tannic acid, γ-oryzanol and other blood-circulation promoters; glycyrrhizinic acid derivatives, glycyrrhetinic acid derivatives, azulene and other anti-inflammatory agents; arginine, serine, leucine, tryptophane and other amino acids; and maltose sucrose condensate, lysozyme chloride and other agents for controlling normal bacteria flora.

Further examples of substances that can be added include, among others, chamomile extract, parsley extract, beech extract, wine yeast extract, grapefruit extract, Japanese honeysuckle extract, rice extract, grape extract, hop extract, rice bran extract, loquat extract, cork tree bark extract, coix seed extract, swertia japonica extract, melilot extract, birch extract, licorice extract, peony extract, soapwort extract, loofa extract, cayenne pepper extract, lemon extract, gentian root extract, perilla extract, aloe extract, rosemary extract, sage extract, thyme extract, tea extract, seaweed extract, cucumber extract, clove extract, carrot extract, horse chestnut extract, hamamelis extract, mulberry extract and various other extracts.

The present invention is explained in further details below using examples. It should be noted, however, that the present invention is not at all limited to these examples.

<Preparation of Agents and Test Method>

[Preparation of Compound Solutions Used for Evaluation Test]

Soybean saponin (Wako Pure Chemical Industries, Ltd.), retinoic acid (all-trans-retinoic acid; Wako Pure Chemical Industries, Ltd.), retinol (all-trans-retinol; Wako Pure Chemical Industries, Ltd.) and silybin (Sigma-Aldrich Corp.) were dissolved in a dimethyl sulfoxide of biochemical reagent grade (DMSO; Wako Pure Chemical Industries, Ltd.), and an appropriate amount of the obtained solution was added to a culture solution and the culture solution was used to treat skin fibroblasts and three-dimensional skin models.

Root of Bletilla striata was cut into thin slices and 100 g of thinly sliced Bletilla striata root was extracted under hot water using a high-speed solvent extractor (ASE-200; Japan Dionex Co., Ltd.). The obtained extract was freeze-dried and condensed to obtain 5.2 g of extract. Water was then added to dissolve this extract until the extract content became 1%. The obtained extract is hereinafter referred to as “Bletilla striata extract.”

Leaves of Iris sanguinea and aril of Euphoria longan (longan fruit) were cut into thin slices and 100 g each of thinly sliced Iris sanguinea leaves and longan fruit were extracted under ethanol (99.5%) using a high-speed solvent extractor (ASE-200; Japan Dionex Co., Ltd.). The resulting extracts were condensed to obtain 10.5 g and 11.3 g of extracts, respectively. 50% 1,3-butylene glycol was then added to dissolve these extracts until the extract content became 1% each. The obtained extracts are hereinafter referred to as “Iris sanguinea extract” and “Euphoria longan extract,” respectively.

[Culturing of Normal Human Skin Fibroblasts]

Normal human skin fibroblasts (hereinafter referred to as “NFB”) CCD1059 (purchased from Dainippon Pharmaceutical Co., Ltd.) was cultured in a skin fibroblast culture medium FGM (Sanko Junyaku Co., Ltd.) in an incubator adjusted to 37° C. and 5% CO₂. FGM was prepared by adding a human fibroblast growth factor (1 μg/ml), insulin (5 mg/ml), gentamycin (50 μg/ml) and amphotericin B (50 μg/ml) to a fibroblast basal medium. Cells from third through seventh passages were used in this test.

[Preparation of NFB Culture Supernatant and NFB Extract Treated with Each Agent]

NFB was inoculated into a cell culture dish of 90 mm in diameter and cultured until a 90% confluent was obtained. The culture medium was then changed to FGM in which each agent was added, and the medium was cultured for 24 hours. The culture solution was then discarded and NFB was washed with 1×PBS (−) (phosphoric acid buffer saline solution not containing calcium or magnesium), after which FGM was added and UVA was irradiated at 10 J/cm². UVA was irradiated for 30 minutes at a UV intensity of 5.55 W/cm² using FL20S-BL/DMR (Clinical Supply Co., Ltd.) to achieve a total irradiation dose of 10 J/cm². UV intensity was measured using a UV MONITOR MS-211-I (Eiko Instruments Co., Ltd.).

The culture medium was again changed to FGM in which each agent was added, and the medium was cultured for 24 hours. As a control group, NFB samples not irradiated with UVA were also prepared.

Supernatant samples of NFB cultures treated with various agents were prepared as follows. The supernatant of the NFB culture treated with each agent was collected and centrifuged at 1,200×G for 5 minutes to remove suspended cells, after which another round of centrifuging was performed at 15,000×G for 15 minutes to remove cell fragments. The obtained solution was dialyzed in water and then freeze-dried, followed by a dissolution in 20 mM Tris-HCl (pH 7.5) to obtain a ×50 concentrate. This concentrate was used as a culture supernatant sample.

Samples of NFB cell extracts treated with various agents were prepared as follows. After the culture supernatant was collected, the cells were washed with PBS (−) and then a cell extraction solution (20 mM Tris-HCl (pH 7.5) containing 0.4% Nonidet P-40) was added and the mixture was agitated for 30 minutes at 4° C. The cell extract was then collected, dialyzed in water, freeze-dried, and then dissolved in a cell extraction solution to obtain a ×20 concentrate. This concentrate was used as a fibroblast extract sample.

[Culturing of Three-Dimensional Human Skin Model]

Three-dimensional human skin models are widely used in safety evaluation and effectiveness evaluation as a simulated model of human skin. In this test, TESTSKIN (LSE-high) (Toyobo Co., Ltd.) was used as a three-dimensional human skin model.

[Preparation of Three-Dimensional Human Skin Model Extract Treated with Each Agent]

A culture medium was added to the outer well of TESTSKIN (LSE-high) and cultured for 24 hours. UVA and UVB were irradiated at 10 J/cm² and 100 mJ/cm², respectively. UVB was irradiated for 2 minutes at a UV intensity of 0.83 W/cm² using FL20S-E-30/DMR (Clinical Supply Co., Ltd.) to achieve a total irradiation dose of 100 mJ/cm². UV intensity was measured using a UV MONITOR MS-211-I (Eiko Instruments Co., Ltd.).

As a control group, three-dimensional human skin models not irradiated with UVA or UVB were also prepared.

Thereafter, the culture solution was changed and each agent was added by 100 μl onto the tissues in the assay ring, after which the medium was cultured for 36 hours. Thereafter, the tissues were collected and a tissue extraction solution (50 mM Tris-HCl (pH 7.5), 0.5% (Octylphenoxy) polyethoxyethanol (Sigma-Aldrich Corp.)) was added, and then the mixture was homogenized using a Teflon (registered trademark) homogenizer. The homogenized mixture was centrifuged for 30 minutes at 10,000×G to remove tissue fragments, and then dialyzed overnight in distilled water at 4° C. Thereafter, the dialyzed mixture was freeze-dried to remove water. A tissue extraction solution was added to obtain a ×20 concentrate, and this concentrate was used as a three-dimensional skin model extract sample.

[Measurement of Carbonylated Protein]

Carbonylated protein is a type of oxidized protein and known as a bioindicator of aging of a living organism. By measuring carbonylated protein in a living organism treated with an agent, the aging suppression action of the agent can be tested (Chiryogaku (Biomedicine & Therapeutics), Vol. 32, No. 4, pp. 58-61, 1998). For example, the aging suppression action of an agent can be tested by irradiating UV light onto NFB treated with the agent and then measuring the carbonylated protein level in the cells using a known method (Nakamura, et al., Journal of Biochemistry, Vol. 199, pp. 768-774, 1996).

In this test, 2,4-dinitrophenyl hydrazine (DNPH), which specifically bonds with the carbonyl group in the protein produced by oxidation damage, was used to label carbonylated protein, and then detection was performed using an anti-DNPH antibody that specifically bonds with DNPH.

The specific method is explained below. DNPH protein was detected from the protein in the sample by means of Western blotting using a DNPH kit (Oxi Blot™ Protein Oxidation Detection Kit; Chemicon International Inc.). Detection was performed by exposing PVDF membranes to light using a fluorescence detection kit (ECL PLUS; Amarsham PLC), and an automatic developer (FPM100; FUJIFILM Medical Co., Ltd.) was used to transfer images. A densitometer (Molecular Dynamics Inc.) was then used to analyze the images to quantify the photographic density. For your information, protein in the sample was reduced and measured as deemed appropriate. In this case, 2-mercaptoethanol was used as a reducing agent and the mixture was heated for 5 minutes at 100° C. to sever the disulfide bond.

[Immunoprecipitation of Type I Collagen]

Using the protein derived from the three-dimensional human skin model extract treated with each agent, type I collagen was immunoprecipitated by means of a known method (Mizushima, et al., Jpn. J. Cancer Res. Vol. 93, pp. 652-659, 2002). Specifically, 20 of the protein derived from the three-dimensional human skin model extract treated with each agent was mixed with a STEN buffer solution (50 mM Tris-HCl (pH 7.5), 150 mM NaCl, 1 mM EDTA, 0.2% Nonidet P-40) until the total volume became 500 μl, and then a polyclonal antibody (Rockland Inc.) for immunoprecipitation of type I collagen was added to achieve the final concentration of 1 μg/ml. The obtained mixture was agitated for one whole day at 4° C. to cause reaction, after which 20 μg of sepharose 4B (ICN Pharmaceuticals, Inc.) that had been caused to bond with anti-rabbit immunoglobulin G was added and the mixture was agitated for 2 hours at 4° C. to cause reaction. The immunoprecipitate was washed three times using a STEN buffer solution, after which 50 μl of a SDS-PAGE sample buffer solution was added and the amount of carbonylated protein and amount of type I collagen were measured by means of Western blotting.

[Measurement of Proteasome Activity]

Proteasome activity was measured using a known method (Hayashi, et al., Mechanisms of Aging and Development, Vol. 102, pp. 55-66, 1998) based on cell extract samples.

The specific method is as follows. For the matrix to measure trypsin-like proteasome activity, t-butyloxycarbonyl-L-leucyl-L-arginyl-L-arginyl-L-4-methyl-coumaryl-7-amide (Peptide Institute, Inc.) was used. A cell extract sample containing protein by an equivalent of 10 μg was added to 10.5 μl of a 100 μM matrix solution prepared with 100 mM Tris-HCl (pH 8.0), after which a cell extraction solution was added to a total volume of 50 μl. The obtained solution was treated for 30 minutes at 37° C., after which the fluorescence intensity of the separated 7-amine-4-methyl coumarin was measured at an excitation wavelength (Ex) of 380 nm and absorption waveform (Em) of 440 nm. The result was obtained for each sample type treated with each agent, as an “average ±standard error” calculated from the measured values of three samples.

### Example 1

[Evaluation of Action to Suppress Accumulation of Carbonylated Protein]

As a result of evaluating 20 types of plant extracts for their action to suppress accumulation of carbonylated protein, Bletilla striata extract and Iris sanguinea extract exhibited a notable effect. The results of Bletilla striata extract and Iris sanguinea extract that were found to have the aforementioned effect are shown, as well as the result of Euphoria longan extract as an example of a plant extract that did not have such effect. Skin fibroblasts were treated with various plant extracts (extract content: 1%) by 1% (final extract concentration: 0.01%) and the amount of carbonylated protein was measured under conditions with and without UVA irradiation by means of Western blotting.

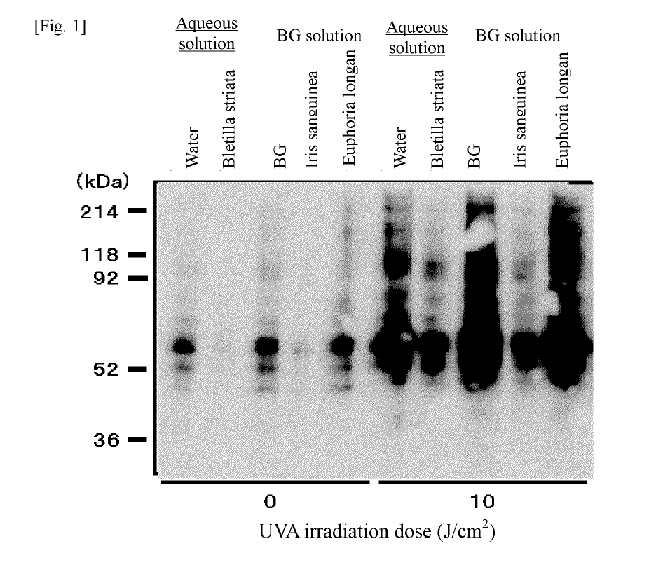

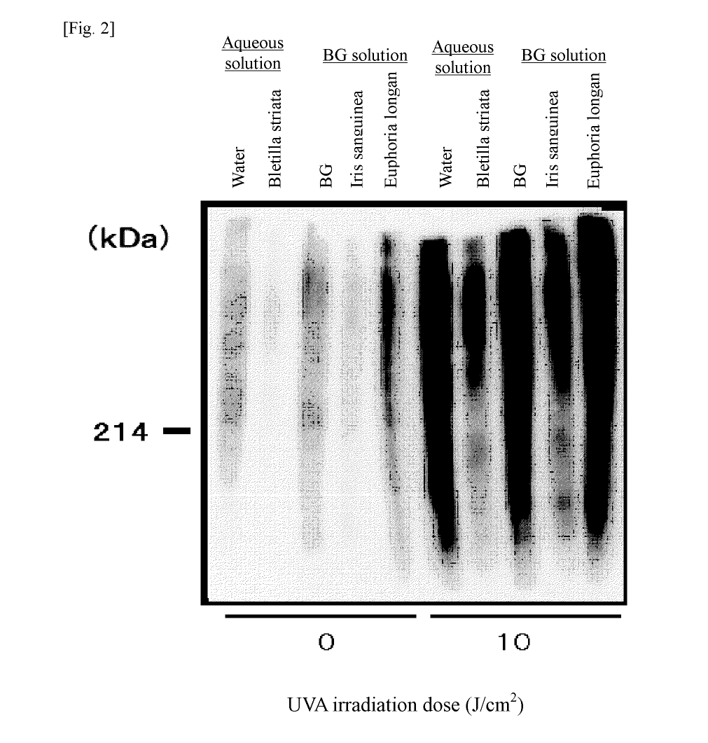

UVA irradiation promoted carbonylation of protein in cells and culture supernatant. When Bletilla striata extract and Iris sanguinea extract were added, accumulation of carbonylated protein in cells (FIG. 1 and Table 1; results of reduced protein [2-mercaptoethanol was added to the protein and the mixture was heated for 5 minutes at 100° C. to sever the disulfide bond]), as well as carbonylated protein in culture supernatant (FIG. 2 and Table 2; results of non-reducing protein [without reduction]), was suppressed regardless of whether or not UV light was irradiated. For your information, the relative photographic densities shown in Tables 1 and 2 are relative values based on the photographic density (amount of carbonylated protein) in a sample treated with water and given no UV irradiation, being 100%. Also for your information, Table 1 shows the numerical values of the results in FIG. 1 obtained by numerical conversion based on image processing. Similarly, Tables 2 through 8 show the numerical values of the results in FIGS. 2 through 8.

TABLE 1

| | | Relative photographic | |
|---|---|---|---|
| | Treatment | density (%) | |
| Agent type | concentration (%) | UV (−) | UV (+) |
| Water | 1 | 100 | 940 |
| Bletilla striata extract | 1 | 5 | 210 |
| BG | 1 | 125 | 1420 |
| Iris sanguinea extract | 1 | 10 | 185 |
| Euphoria longan extract | 1 | 120 | 1150 |

As evident from the results of carbonylated protein (with reduction) in cells as shown in FIG. 1 and Table 1, treating with Bletilla striata extract reduced the amount of carbonylated protein to 5%, compared to the sample not given any treatment (sample treated with water), under the condition without UV irradiation. Also, treating with Iris sanguinea extract reduced the amount of carbonylated protein to 8%, compared to the sample not given any treatment (sample treated with BG). Furthermore, giving UV irradiation produced carbonylated protein and the accumulated amount of carbonylated protein increased to 9 times and 11 times, respectively, in the samples not given any treatment (one treated with water and the other treated with BG), compared to when UV irradiation was not given. Here, treating with Bletilla striata extract reduced the amount of carbonylated protein to 22% compared to the sample not given any treatment (sample treated with water), while treating with Iris sanguinea extract reduced the amount of carbonylated protein to 13% compared to the sample not given any treatment (sample treated with BG).

TABLE 2

| | | Relative photographic | |
|---|---|---|---|
| | Treatment | density (%) | |
| Agent type | concentration (%) | UV (−) | UV (+) |
| Water | 1 | 100 | 1200 |
| Bletilla striata extract | 1 | 5 | 250 |
| BG | 1 | 105 | 1210 |
| Iris sanguinea extract | 1 | 10 | 325 |
| Euphoria longan extract | 1 | 135 | 1460 |

As evident from the results of carbonylated protein (without reduction) in culture supernatant as shown in FIG. 2 and Table 2, treating with Bletilla striata extract reduced the amount of carbonylated protein to 5%, compared to the sample not given any treatment (sample treated with water), under the condition without UV irradiation. Also, treating with Iris sanguinea extract reduced the amount of carbonylated protein to 10%, compared to the sample not given any treatment (sample treated with BG). Furthermore, giving UV irradiation produced carbonylated protein and the accumulated amount of carbonylated protein increased to 12 times in both samples not given any treatment (one treated with water and the other treated with BG), compared to when UV irradiation was not given. Here, treating with Bletilla striata extract reduced the amount of carbonylated protein to 21% compared to the sample not given any treatment (sample treated with water), while treating with Iris sanguinea extract reduced the amount of carbonylated protein to 27% compared to the sample not given any treatment (sample treated with BG).

Although UV irradiation produced carbonylated protein, subsequently adding Bletilla striata extract and Iris sanguinea extract and culturing the mixture caused carbonylated protein to decrease in both cells and supernatant of cells. Accordingly, Bletilla striata extract and Iris sanguinea extract are considered to have an effect of removing abnormal protein that has been produced. On the other hand, such effect of suppressing the accumulation of abnormal protein was not found in Euphoria longan extract. Under both conditions with and without UV irradiation, causing Bletilla striata extract and Iris sanguinea extract to act upon samples reduced carbonylated protein to approx. 1/10th to ¼th compared to the samples not given any treatment, which suggests that these extracts not only suppress production of carbonylated protein, but they also break down or remove produced carbonylated protein. Similar trends are also seen in the test results explained below.

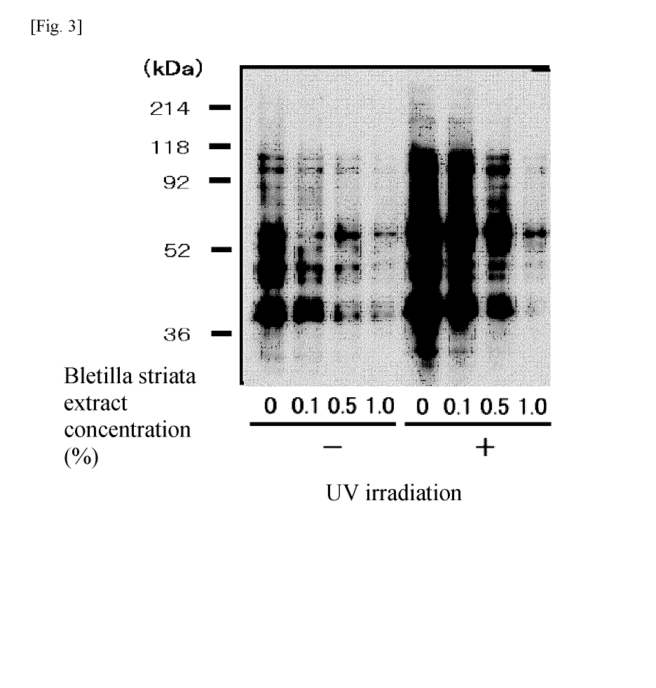

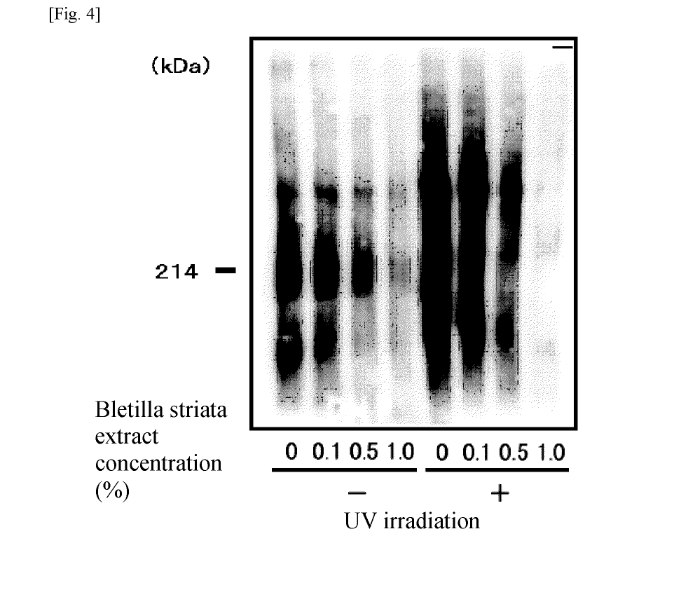

When a three-dimensional human skin model was treated with Bletilla striata extract (extract content: 1%) by 0.1%, 0.5% and 1%, respectively (final extract concentrations: 0.001%, 0.005% and 0.01%, respectively), accumulation of carbonylated protein was suppressed in a manner dependent on the concentration (FIG. 3 and Table 3; results of reduced protein/FIG. 4 and Table 4; results of non-reduced protein). For your information, the relative photographic densities shown in Tables 3 and 4 are relative values based on the photographic density (amount of carbonylated protein) in a sample not treated with Bletilla striata extract (treatment concentration: 0%) nor given UV irradiation, being 100%.

TABLE 3

| | |
|---|---|
| Bletilla striata extract | Relative photographic density (%) |

| treatment concentration (%) | UV (−) | UV (+) |
|---|---|---|
| 0 | 100 | 560 |
| 0.1 | 45 | 315 |
| 0.5 | 25 | 85 |
| 1.0 | 10 | 15 |

As evident from the results of reduced protein as shown in FIG. 3 and Table 3, the accumulated amount of carbonylated protein decreased to 45%, 25% and 10% when a sample was treating with Bletilla striata extract by 0.1%, 0.5% and 1.0%, respectively, compared to the sample not given any treatment, under the condition without UV irradiation. Also, while UV irradiation increased the amount of carbonylated protein to 5.6 times (sample not given any treatment) compared to when UV irradiation was not given, treating with Bletilla striata extract by 0.1%, 0.5% and 1.0% reduced the amount of carbonylated protein to 56%, 15% and 3%, respectively, compared to the sample not given any treatment under UV irradiation.

TABLE 4

| | |
|---|---|
| Bletilla striata extract | Relative photographic density (%) |

| treatment concentration (%) | UV (−) | UV (+) |
|---|---|---|
| 0 | 100 | 620 |
| 0.1 | 63 | 385 |
| 0.5 | 37 | 85 |
| 1.0 | 9 | 10 |

As evident from the results of non-reduced protein as shown in FIG. 4 and Table 4, the accumulated amount of carbonylated protein decreased to 63%, 37% and 9% when a sample was treating with Bletilla striata extract by 0.1%, 0.5% and 1.0%, respectively, compared to the sample not given any treatment, under the condition without UV irradiation. Also, while UV irradiation increased the amount of carbonylated protein to 6.2 times (sample not given any treatment) compared to when UV irradiation was not given, treating with Bletilla striata extract by 0.1%, 0.5% and 1.0% reduced the amount of carbonylated protein to 62%, 14% and 2%, respectively, compared to the sample not given any treatment under UV irradiation.

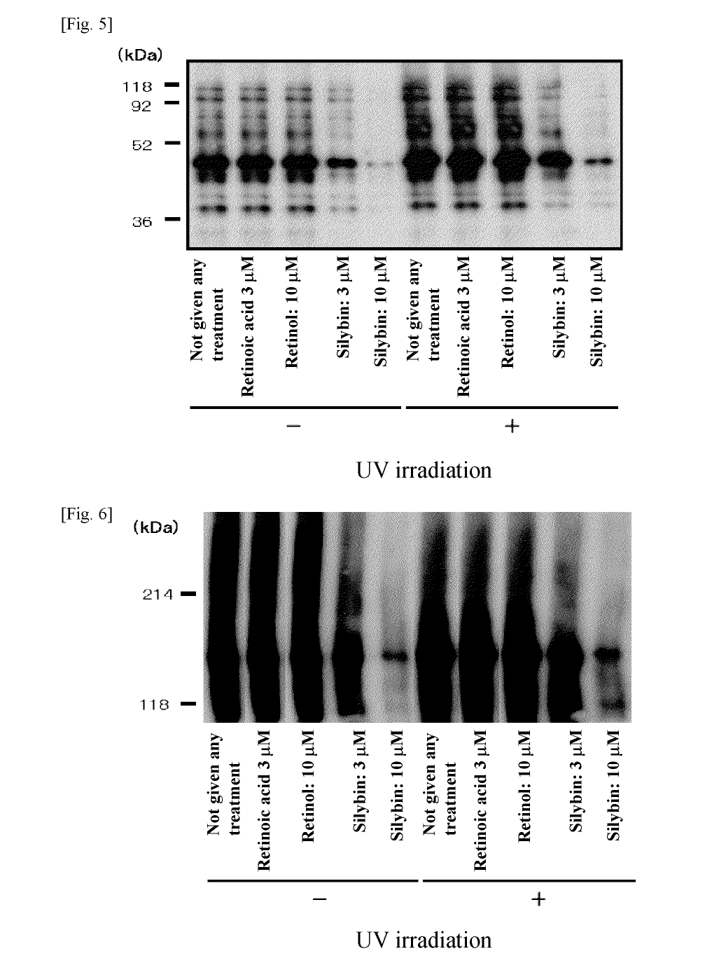

Next, the actions of silybin and forms of vitamin A to suppress accumulation of carbonylated protein were evaluated. Since silybin exhibits similar actions shown by retinoic acid, retinol and other forms of vitamin A, such as suppression of epidermal keratinocyte differentiation and promotion of type I collagen production, retinoic acid and retinol were used as controls. When a three-dimensional human skin model was treated with silybin at 3 and 10 μM, concentrations at which cell toxicity does not occur, accumulation of carbonylated protein was suppressed in a manner dependent on the concentration (FIG. 5 and Table 5; results of reduced protein/FIG. 6 and Table 6; results of non-reduced protein). On the other hand, treating with retinoic acid and retinol at 3 and 10 μM, concentrations at which cell toxicity does not occur, had no effect on accumulation of carbonylated protein as in the sample not given any treatment. For your information, the relative photographic densities shown in Tables 5 and 6 are relative values based on the photographic density (amount of carbonylated protein) in a sample not treated with any agent nor given UV irradiation, being 100%.

TABLE 5

| | | Relative photographic | |
|---|---|---|---|
| | Treatment | density (%) | |
| Agent type | concentration (μM) | UV (−) | UV (+) |
| Not given any treatment | 0 | 100 | 335 |
| Retinoic acid | 3 | 105 | 315 |
| Retinol | 10 | 110 | 330 |
| Silybin | 3 | 50 | 85 |
| Silybin | 10 | 5 | 35 |

To be specific, as evident from the results of reduced protein as shown in FIG. 5 and Table 5, the accumulated amount of carbonylated protein decreased to 50% and 10% compared to the sample not given any treatment, when a sample was treated with silybin at 3 μM and 10 μM, respectively, under the condition without UV irradiation. On the other hand, treating with 3 μM of retinoic acid or 10 μM of retinol caused little change in the amount of carbonylated protein compared to the sample not given any treatment. Also, while UV irradiation increased the amount of carbonylated protein to 3.4 times (sample not given any treatment) compared to when UV irradiation was not given, treating with silybin at 3 μM and 10 μM reduced the amount of carbonylated protein to 25% and 10%, respectively, compared to the sample not given any treatment under UV irradiation. On the other hand, treating with 3 μM of retinoic acid or 10 μM of retinol caused little change in the amount of carbonylated protein compared to the sample not given any treatment.

TABLE 6

| | | Relative photographic | |
|---|---|---|---|
| | Treatment | density (%) | |
| Agent type | concentration (μM) | UV (−) | UV (+) |
| Not given any treatment | 0 | 100 | 320 |
| Retinoic acid | 3 | 110 | 305 |
| Retinol | 10 | 115 | 315 |
| Silybin | 3 | 55 | 95 |
| Silybin | 10 | 8 | 30 |

As evident from the results of non-reduced protein as shown in FIG. 6 and Table 6, the accumulated amount of carbonylated protein decreased to 55% and 8% compared to the sample not given any treatment, when a sample was treated with silybin at 3 μM and 10 μM, respectively, under the condition without UV irradiation. On the other hand, treating with 3 μM of retinoic acid or 10 μM of retinol caused little change in the amount of carbonylated protein compared to the sample not given any treatment. Also, while UV irradiation increased the amount of carbonylated protein to 3.2 times (sample not given any treatment) compared to when UV irradiation was not given, treating with silybin at 3 μM and 10 μM reduced the amount of carbonylated protein to 30% and 9%, respectively, compared to the sample not given any treatment under UV irradiation. On the other hand, treating with 3 μM of retinoic acid or 10 μM of retinol caused little change in the amount of carbonylated protein compared to the sample not given any treatment.

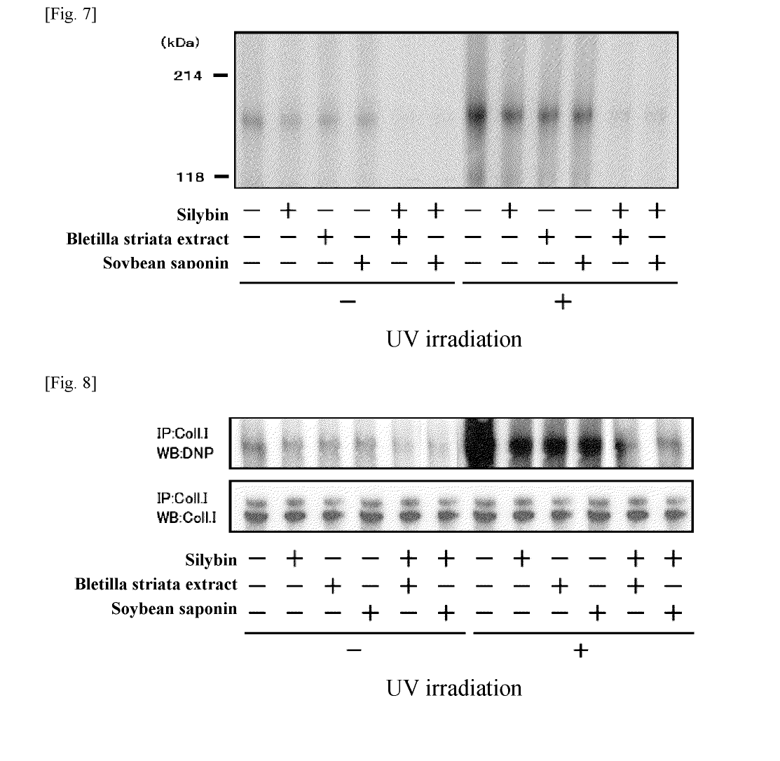

Based on the above results, it is clear that Bletilla striata extract and silybin suppress accumulation of carbonylated protein in a three-dimensional skin model in a manner dependent on the concentration. Accordingly, whether or not a combined use of Bletilla striata extract and silybin would synergistically suppress accumulation of carbonylated protein was evaluated. Also, whether or not a combined use of silybin and soybean saponin, a substance known to have an action to suppress accumulation of carbonylated protein, would synergistically suppress accumulation of carbonylated protein was evaluated. As a result, regardless of whether or not UV light was irradiated using silybin (3 μM) in combination with Bletilla striata extract (0.1%; final extract concentration of 0.001%), or using silybin (3 μM) in combination with soybean saponin (0.0005%), suppressed the accumulation of carbonylated protein synergistically compared to when a sample was treated with silybin (3 μM), Bletilla striata extract (0.1%; final extract concentration of 0.001%) or soybean saponin (0.0005%) alone, as far as accumulation of carbonylated protein in a three-dimensional skin model was concerned (FIG. 7 and Table 7; results of non-reduced protein). For your information, the relative photographic densities shown in Table 7 are relative values based on the photographic density (amount of carbonylated protein) in a sample not treated with any agent nor given UV irradiation, being 100%. The amount of each agent shown in FIG. 7 is the same as the corresponding amount listed in Table 7.

TABLE 7

| | Silybin | Bletilla striata | Soybean | Relative photographic density (%) | |
|---|---|---|---|---|---|
| Agent type | (μM) | extract (%) | saponin (%) | UV (−) | UV (−) |
| Not given any | 0 | 0 | 0 | 100 | 500 |
| treatment | | | | | |
| Silybin | 3 | 0 | 0 | 60 | 300 |
| Bletilla striata | 0 | 0.1 | 0 | 75 | 350 |
| extract | | | | | |
| Soybean saponin | 0 | 0 | 0.0005 | 72 | 365 |
| Silybin + | 3 | 0.1 | 0 | 10 | 50 |
| Bletilla striata | | | | | |
| extract | | | | | |
| Silybin + | 3 | 0 | 0.0005 | 11 | 55 |
| soybean saponin | | | | | |

To be specific, as evident from the results of non-reduced protein as shown in FIG. 7 and Table 7, the accumulated amount of carbonylated protein decreased to 60%, 75% and 72% compared to the sample not given any treatment, when a sample was treated with 3 μM of silybin, 0.1% of Bletilla striata extract and 0.0005% of soybean saponin, respectively, under the condition without UV irradiation. Furthermore, a combined use of 3 μM of silybin and 0.1% of Bletilla striata extract reduced the amount of carbonylated protein to 10%. Also, a combined use of 3 μM of silybin and 0.0005% of soybean saponin reduced the amount of carbonylated protein to 11%. These figures indicate synergistic effects.

UV irradiation increased the amount of carbonylated protein to 5 times (sample not given any treatment) compared to when UV irradiation was not given, but treating with 3 μM of silybin, 0.1% of Bletilla striata extract and 0.0005% of soybean saponin caused the amount of carbonylated protein to decrease to 60%, 70% and 73%, respectively, compared to the sample not given any treatment under UV irradiation. Also, a combined use of 3 μM of silybin and 0.1% of Bletilla striata extract reduced the amount of carbonylated protein to 10% compared to the sample not given any treatment under UV irradiation. Furthermore, a combined use of 3 μM of silybin and 0.0005% of soybean saponin reduced the amount of carbonylated protein to 11% compared to the sample not given any treatment under UV irradiation. These figures indicate synergistic effects.

From the results shown in FIG. 7 and Table 7, it is clear that using silybin in combination with Bletilla striata extract, or using silybin in combination with soybean saponin, synergistically suppresses carbonylated protein. Next, whether or not carbonylation of type I collagen would be suppressed by using silybin in combination with Bletilla striata extract, or using silybin in combination with soybean saponin, was evaluated to check the possibility of using such combinations to remove oxidized collagen that accumulates with age to cause lines, sagging, dull complexion and other signs of aging skin. To be specific, protein contained in a three-dimensional skin model extract was immunoprecipitated using a type I collagen antibody and carbonylated collagen was detected by means of Western blotting.

The top graph in FIG. 8 shows the detection result of carbonylated collagen by means of Western blotting (WB) using a DNP antibody after an immunoprecipitation (IP) using a type I collagen antibody. The bottom graph in FIG. 8 shows the detection result of immunoprecipitated carbonylated collagen by means of Western blotting (WB) using a type I collagen antibody, after an immunoprecipitation (IP) using a type I collagen antibody. The amount of immunoprecipitated type I collagen was identical among the samples treated with different agents and regardless of whether or not UV light was irradiated. Accordingly, the amount of carbonylated collagen detected in the top graph in FIG. 8 does not depend on the collagen amount, but it depends on the carbonylation level of collagen.

Regardless of whether or not UV light was irradiated, using silybin (3 μM) in combination with Bletilla striata extract (0.1%; final extract concentration of 0.001%), or using silybin (3 μM) in combination with soybean saponin (0.0005%), suppressed the accumulation of carbonylated collagen synergistically compared to when a sample was treated with silybin (3 μM), Bletilla striata extract (0.1%; final extract concentration of 0.001%) or soybean saponin (0.0005%) alone, as far as accumulation of carbonylated collagen in a three-dimensional skin model was concerned (FIG. 8 and Table 8; results of non-reduced protein). For your information, the relative photographic densities shown in Table 8 are relative values based on the photographic density (amount of carbonylated protein) in a sample not treated with any agent nor given UV irradiation, being 100%. The amount of each agent shown in FIG. 8 is the same as the corresponding amount listed in Table 8.

TABLE 8

| | Silybin | Bletilla striata | Soybean | Relative photographic density (%) | |
|---|---|---|---|---|---|
| Agent type | (μM) | extract (%) | saponin (%) | UV (−) | UV (−) |
| Not given any | 0 | 0 | 0 | 100 | 600 |
| treatment | | | | | |
| Silybin | 3 | 0 | 0 | 72 | 430 |
| Bletilla striata | 0 | 0.1 | 0 | 83 | 480 |
| extract | | | | | |
| Soybean saponin | 0 | 0 | 0.0005 | 78 | 450 |
| Silybin + | 3 | 0.1 | 0 | 12 | 75 |
| Bletilla striata | | | | | |
| extract | | | | | |
| Silybin + | 3 | 0 | 0.0005 | 15 | 88 |
| soybean saponin | | | | | |

To be specific, as evident from the results of non-reduced protein as shown in FIG. 8 and Table 8, the accumulated amount of carbonylated collagen decreased to 72%, 83% and 78% compared to the sample not given any treatment, when a sample was treated with 3 μM of silybin, 0.1% of Bletilla striata extract and 0.0005% of soybean saponin, respectively, under the condition without UV irradiation. Furthermore, a combined use of 3 μM of silybin and 0.1% of Bletilla striata extract reduced the amount of carbonylated collagen to 12%. Also, a combined use of 3 μM of silybin and 0.0005% of soybean saponin reduced the amount of carbonylated collagen to 15%. These figures indicate synergistic effects. UV irradiation increased the amount of carbonylated collagen to 6 times (sample not given any treatment) compared to when UV irradiation was not given, but treating with 3 μM of silybin, 0.1% of Bletilla striata extract and 0.0005% of soybean saponin caused the amount of carbonylated collagen to decrease to 72%, 80% and 75%, respectively, compared to the sample not given any treatment under UV irradiation. Also, a combined use of 3 μM of silybin and 0.1% of Bletilla striata extract reduced the amount of carbonylated collagen to 13% compared to the sample not given any treatment under UV irradiation. Furthermore, a combined use of 3 μM of silybin and 0.0005% of soybean saponin reduced the amount of carbonylated collagen to 15% compared to the sample not given any treatment under UV irradiation. These figures indicate synergistic effects.

### Example 2

### Evaluation of Action to Promote Proteasome Activity

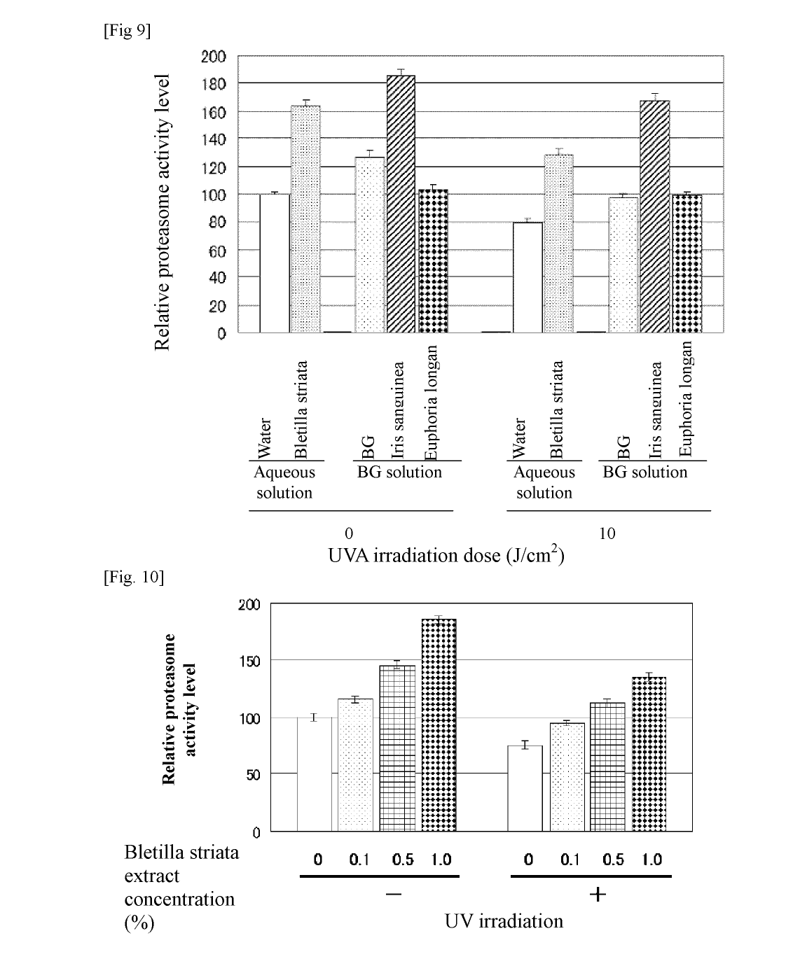

The actions of Bletilla striata extract, Iris sanguinea extract and Euphoria longan extract to promote proteasome activity were evaluated. Skin fibroblasts were treated with various plant extracts (extract content 1%) by 1% (final extract concentration: 0.01%) and proteasome activity was measured under conditions with and without UVA irradiation using the aforementioned method. UVA irradiation caused proteasome activity to drop by approx. 20% compared to when UVA irradiation was not given. By treating with Bletilla striata extract and Iris sanguinea extract, however, proteasome activity was promoted regardless of whether or not UVA irradiation was given (FIG. 9).

To be specific, proteasome activity increased to 163% compared to the sample not given any treatment (sample treated with water), when a sample was treated with Bletilla striata extract, under the condition without UVA irradiation. Also, the relative proteasome activity levels in the sample not given any treatment (sample treated with BG) and sample treated with Iris sanguinea extract were 127% and 185%, respectively, and the proteasome activity in the sample treated with Iris sanguinea extract increased to 146% compared to the sample not given any treatment (sample treated with BG). On the other hand, treating with Euphoria longan extract (relative proteasome activity level: 104%) caused proteasome activity to drop to 82% compared to the sample not given any treatment (sample treated with BG).

UVA irradiation causes proteasome activity to drop. In the two samples not given any treatment (one treated with water and the other treated with BG), proteasome activity dropped to 80% and 97%, respectively. Under UVA irradiation, however, the relative proteasome activity level increased to 129% when a sample was treated with Bletilla striata extract. This is an increase of 161% compared to the sample not given any treatment (sample treated with water) under UVA irradiation (80%). Also, under UVA irradiation the relative proteasome activity level increased to 167% when a sample was treated with Iris sanguinea extract. This is an increase of 172% compared to the sample not given any treatment (sample treated with BG) under UVA irradiation (97%). On the other hand, treating with Euphoria longan extract caused little increase in proteasome activity.

Also, when a three-dimensional human skin model was treated with Bletilla striata extract (extract content: 1%) by 0.1%, 0.5% and 1%, respectively (final extract concentrations: 0.001%, 0.005% and 0.01%, respectively), proteasome activity was promoted in a manner dependent on the concentration (FIG. 10).

To be specific, proteasome activity increased to 115%, 145% and 185% compared to the sample not given any treatment (sample treated with water), when a sample was treated with 0.1%, 0.5% and 1.0% of Bletilla striata extract, respectively, under the condition without UVA irradiation.

UVA irradiation causes proteasome activity to drop. Proteasome activity dropped to 75% in the sample not given any treatment (treated with water). Under UVA irradiation, treating with 0.1%, 0.5% and 1.0% of Bletilla striata extract achieved proteasome activities of 95%, 112% and 135%, respectively. However, these correspond to increases of 127%, 149% and 180%, respectively, when compared to the sample not given any treatment (sample treated with water) whose proteasome activity dropped to 75% under UVA irradiation.

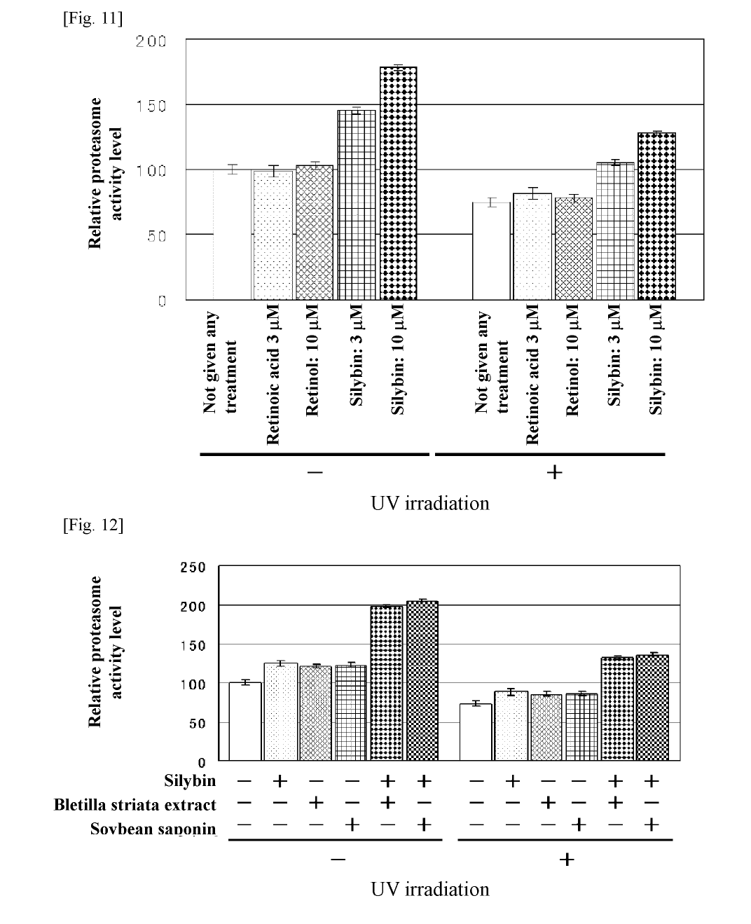

Next, the actions of silybin and forms of vitamin A to promote proteasome activity were evaluated. Since silybin exhibits similar actions shown by retinoic acid, retinol and other forms of vitamin A, such as suppression of epidermal keratinocyte differentiation and promotion of type I collagen production, retinoic acid and retinol were used as controls. When a three-dimensional human skin model was treated with silybin at 3 and 10 μM, concentrations at which cell toxicity does not occur, proteasome activity was promoted in a manner dependent on the concentration (FIG. 11). On the other hand, treating with retinoic acid and retinol at 3 and 10 μM, concentrations at which cell toxicity does not occur, had no effect on proteasome activity as in the sample not given any treatment.

To be specific, proteasome activity increased to 145% and 178% compared to the sample not given any treatment, when a sample was treated with silybin at 3 μM and 10 μM, respectively, under the condition without UV irradiation. On the other hand, treating with 3 μM of retinoic acid or 10 μM of retinol caused little change in proteasome activity compared to the sample not given any treatment. Also, while UV irradiation caused proteasome activity to drop to 75% compared to when UV irradiation was not given, treating with silybin at 3 μM and 10 μM increased the relative proteasome activity level to 105% and 128%, respectively, even under UV irradiation. These correspond to increases of 140% and 170%, respectively, compared to the sample not given any treatment under UV irradiation (75%). On the other hand, treating with 3 μM of retinoic acid or 10 μM of retinol caused little change in proteasome activity compared to the sample not given any treatment.

Based on the above results, it is clear that Bletilla striata extract and silybin promote proteasome activity in a three-dimensional skin model in a manner dependent on the concentration. Accordingly, whether or not a combined use of Bletilla striata extract and silybin would synergistically promote proteasome activity was evaluated. Also, whether or not a combined use of silybin and soybean saponin, a substance known to have an action to promote proteasome activity, would synergistically promote proteasome activity was evaluated. As a result, regardless of whether or not UV light was irradiated, using silybin (3 μM) in combination with Bletilla striata extract (0.1%; final extract concentration of 0.001%), or using silybin (3 μM) in combination with soybean saponin (0.0005%), promoted proteasome activity synergistically compared to when a sample was treated with silybin (3 μM), Bletilla striata extract (0.1%; final extract concentration of 0.001%) or soybean saponin (0.0005%) alone, as far as promotion of proteasome activity in a three-dimensional skin model was concerned (FIG. 12).

To be specific, proteasome activity increased to 125%, 121% and 123% compared to the sample not given any treatment, when a sample was treated with 3 μM of silybin, 0.1% of Bletilla striata extract and 0.0005% of soybean saponin, respectively, under the condition without UV irradiation. Furthermore, a combined use of 3 μM of silybin and 0.1% of Bletilla striata extract increased proteasome activity to 198%. Also, a combined use of 3 μM of silybin and 0.0005% of soybean saponin increased proteasome activity to 205%. These figures indicate synergistic effects.

UV irradiation caused proteasome activity to drop to 73% (sample not given any treatment) compared to when UV irradiation was not given. However, treating with 3 μM of silybin, 0.1% of Bletilla striata extract and 0.0005% of soybean saponin caused the relative proteasome activity level to increase to 88%, 85% and 86%, respectively, corresponding to increases of 121%, 116% and 118%, respectively, compared to the sample not given any treatment under UV irradiation (73%). Also, a combined use of 3 μM of silybin and 0.1% of Bletilla striata extract achieved a relative proteasome activity level of 132%. This corresponds to an increase of 181% compared to the sample not given any treatment under UV irradiation (73%). Furthermore, a combined use of 3 μM of silybin and 0.0005% of soybean saponin achieved a relative proteasome activity level of 136%. This corresponds to an increase of 186% compared to the sample not given any treatment under UV irradiation (73%). These figures indicate synergistic effects.

Example of Prescription 1 [Capsules]

| (Composition) | (Content: mg) |
|---|---|
| Soybean saponin | 25 |
| Milk thistle extract | 25 |
| (containing 35% silybin) | |
| Tocotrienol | 30 |
| Bees wax | 10 |
| Grape seed oil | 110 |

The above ingredients were mixed and the resulting mixture was filled into capsules containing a mixture of gelatin and glycerin to obtain soft capsules.

Example of Prescription 2 [Capsules]

| (Composition) | (Content: mg) |
|---|---|
| Bletilla striata extract | 25 |
| Milk thistle extract | 25 |
| (containing 35% silybin) | |
| Tocotrienol | 30 |
| Bees wax | 10 |
| Grape seed oil | 110 |

The above ingredients were mixed and the resulting mixture was filled into capsules containing a mixture of gelatin and glycerin to obtain soft capsules.

Example of Prescription 3 [Tablets]

| (Composition) | (Content: mg) |
|---|---|
| Soybean saponin | 25 |
| Milk thistle extract | 20 |
| (containing 35% silybin) | |
| Collagen hydrolyzate | 50 |
| Cellulose | 40 |
| Starch | 20 |
| Sucrose fatty acid ester | 2 |

The above ingredients were mixed and compressed into a tablet shape to obtain tablets.

Example of Prescription 4 [Juice]

| | (Content: % |
|---|---|
| (Composition) | by mass) |
| Fructose-glucose liquid sugar | 5.00 |
| Citric acid | 10.40 |
| L-ascorbic acid | 0.20 |
| Aromatic agent | 0.02 |
| Colorant | 0.10 |
| Gelatin decomposition product | 1.00 |
| (average molecular weight: 300) | |
| Soybean saponin | 1.00 |
| Milk thistle extract | 1.00 |
| (containing 35% silybin) | |
| Water | 81.28 |

Example of Prescription 5 [Cream]

| | (Content: % |
|---|---|
| (Composition) | by mass) |
| (1) Stearyl alcohol | 6.0 |
| (2) Stearic acid | 2.0 |
| (3) Hydrogenated lanolin | 4.0 |
| (4) Squalane | 9.0 |
| (5) Octyl dodecanol | 10.0 |
| (6) POE (25) cetyl alcohol ether | 3.0 |
| (7) Glycerin monostearate | 2.0 |
| (8) Gelatin decomposition product | 1.0 |
| (average molecular weight: 300) | |
| (9) Silybin | 1.0 |
| (10) Bletilla striata extract | 0.1 |
| (11) Preservative | As |
| | appropriate |
| (12) Aromatic agent | As |
| | appropriate |
| (13) 1,3-butylene glycol | 6.0 |
| (14) PEG 1500 | 4.0 |
| (15) Purified water | Remainder |

Among the above ingredients, (1) through (12) were dissolved by heating to 80° C. to obtain an oil phase. Ingredients (13) through (15) were dissolved by heating to 70° C. to obtain a water phase. The water phase was gradually added to the oil phase to cause emulsification and the mixture was cooled to 40° C. under agitation, and then further cooled to 30° C. under agitation to obtain cream.

Example of Prescription 6 [Cream]

| | (Content: % |
|---|---|
| (Composition) | by mass) |
| (1) Stearyl alcohol | 6.0 |
| (2) Stearic acid | 2.0 |
| (3) Hydrogenated lanolin | 4.0 |
| (4) Squalane | 9.0 |
| (5) Octyl dodecanol | 10.0 |
| (6) POE (25) cetyl alcohol ether | 3.0 |
| (7) Glycerin monostearate | 2.0 |
| (8) Gelatin decomposition product | 1.0 |
| (average molecular weight: 300) | |
| (9) Silybin | 1.0 |
| (10) Soybean saponin | 1.0 |
| (11) Preservative | As |
| | appropriate |
| (12) Aromatic agent | As |
| | appropriate |
| (13) 1,3-butylene glycol | 6.0 |
| (14) PEG 1500 | 4.0 |
| (15) Purified water | Remainder |

Among the above ingredients, (1) through (12) were dissolved by heating to 80° C. to obtain an oil phase. Ingredients (13) through (15) were dissolved by heating to 70° C. to obtain a water phase. The water phase was gradually added to the oil phase to cause emulsification and the mixture was cooled to 40° C. under agitation, and then further cooled to 30° C. under agitation to obtain cream.

We claim:

1. A method for treating symptoms or diseases associated with accumulation of abnormal protein in the body, comprising administering to a subject desiring such treatment a composition containing silybin and soybean saponin in an amount effective to reduce or remove abnormal protein produced in the body.

2. The method according to claim 1, wherein the administered amount of the composition is an amount effective to promote proteasome activity in the body.

3. The method according to claim 1, wherein the abnormal protein is protein oxidized due to exposure to UV light.

4. The method according to claim 3, wherein the abnormal protein is carbonylated collagen.

5. The method according to claim 1, wherein the administration of the composition is topical administration.

6. The method according to claim 1, wherein the administration of the composition is oral administration.

7. The method according to claim 1, wherein the symptoms or diseases are increases of lines, sagging, dull complexion and/or pigmentation of skin.

8. The method according to claim 5, wherein the composition contains silybin and soybean saponin in an amount of 0.01 to 2% by weight.

9. The method according to claim 6, wherein the composition contains silybin and soybean saponin in an amount of 0.1 to 10% by weight.

10. The method according to claim 1, wherein the composition contains silybin and soybean saponin in an amount synergistically effective to reduce or remove abnormal protein produced in the body, as compared with the silybin alone or the soybean saponin alone.

11. The method according to claim 1, wherein the composition further contains collagen and/or gelatin decomposition products.

12. A method for improving lines, sagging, dull complexion and/or pigmentation of skin, comprising reducing or removing abnormal protein produced in the body by administering to a subject desiring such improvements of skin a composition containing silybin and soybean saponin in an amount effective to reduce or remove abnormal protein in human skin fibroblasts.

13. The method according to claim 12, wherein the abnormal protein is carbonylated collagen.

14. The method according to claim 12, wherein the administration of the composition is topical administration.

15. The method according to claim 12, wherein the administration of the composition is oral administration.
